(19) United States
(12) Patent Application Publication
(10) Pub. No.: US 2022/0031565 A1
(43) Pub. Date: Feb. 3, 2022

(54) DEVICE AND METHOD FOR THREE-DIMENSIONALLY MAPPING ACUPUNCTURE POINTS

(71) Applicant: Yun Kyoung YIM, Daejeon (KR)

(72) Inventor: Yun Kyoung YIM, Daejeon (KR)

(21) Appl. No.: 17/499,823

(22) Filed: Oct. 12, 2021

Related U.S. Application Data
(63) Continuation-in-part of application No. 16/348,141, filed on May 7, 2019.

(30) Foreign Application Priority Data
Mar. 21, 2017 (KR) 10-2017-0035072

(51) Int. Cl. A61H 39/02; A61H 39/00; G16H 30/40; G06T 17/00

(52) U.S. Cl. CPC A61H 39/02; A61H 39/002; A61H 2201/5043; G06T 17/00; G06T 2219/2016; G16H 30/40

(57) ABSTRACT

A three-dimensional acupoint mapping method executable by a computing device is disclosed. The present invention comprises the steps of: retrieving a standard template having a 3D data of human organ and a standard position of an acupoint; transforming coordinate system of the standard template as a 3D image of a patient including coordinates of the patient's organ corresponding to the human organ of the standard template is input; retrieving coordinates of the standard position of the acupoint from the transformed coordinate system of the standard template; and calculating a patient's acupoint's coordinates by mapping the retrieved coordinates of the standard position of the acupoint to the 3D image of a patient.

### CROSS-REFERENCE TO PRIOR APPLICATIONS

This application is a CIP application of U.S. patent application Ser. No. 16/348/141, filed on May 7, 2019, which is national Stage Patent Application of PCT International Patent Application No. PCT/KR2018/002291, filed on Feb. 23, 2018 under 35 U.S.C. § 371, which claims priority of Korean Patent Application No. 10-2017-0035072, filed on Mar. 21, 2017, which are all hereby incorporated by reference in their entirety.

### FIELD OF THE INVENTION

The present invention relates to a three-dimensional (3D) acupoint mapping device and method.

### BACKGROUND OF THE RELATED ART

Acupoints are located on the meridians of the human body to perform acupuncture or moxibustion thereon in traditional eastern medicine, and many technologies for providing the locations of acupoints have been known in conventional practices.

However, the conventional technologies only provide the superficial locations of acupoints on the body surface or the internal locations of acupoints on the basis of a three-dimensional sample made to computer graphics, and accordingly, they do not provide accurate three-dimensional acupoint locations or meridian pathways throughout the human body. Besides, they do not provide accurate information on the safe direction and depth of needle insertion in acupuncture on the basis of the anatomical structures around the acupoint and the acupuncture skill level of an acupuncture practitioner.

When the conventional technologies determine or provide the locations of acupoints, further, they depend upon the self-determination of the practitioner or traditional literatures on acupuncture to provide the locations of acupoints on the surface of a prefabricated fixed sample, and accordingly, they do not consider differences between the anatomical states of individual patients and differences between the acupuncture skill levels of practitioners, thereby unfortunately failing to provide three-dimensional acupoint locations customized to the individual patients and safe acupuncture information customized to the acupuncture skill levels of practitioners.

In addition, most of conventional diagnostic devices of meridian and acupoint are based on physiological characteristics of the acupoints on the body surface, and accordingly, they do not reflect biometric information on acupoints in the deep body.

### DETAILED EXPLANATION OF THE INVENTION

### Technical Problems

Accordingly, the present invention has been made in view of the above-mentioned problems occurring in the prior art, and it is an object of the present invention to provide a three-dimensional acupoint mapping device and method that provides three-dimensional acupoint locations or meridian pathways in the deep body on the basis of real human body images and provides accurate information on safe direction and depth of needle insertion in acupuncture on the basis of the anatomical structures in the human body.

It is another object of the present invention to provide a three-dimensional acupoint mapping device and method that considers differences between the anatomical states of individual patients and differences between the acupuncture skill levels of practitioners to provide three-dimensional acupoint locations customized to the individual patients and safe acupuncture information customized to the acupuncture skill levels of practitioners.

It is yet another object of the present invention to provide a three-dimensional acupoint mapping device and method that provides three-dimensional meridian and acupoint information to develop a diagnostic device of meridian and acupoint using various biometric information (blood flow speed, the number of blood cells, ion concentration, concentration of specific protein, and so on) in the deep body as well as on the body surface around an acupoint.

It is still another object of the present invention to provide a three-dimensional acupoint mapping device and method that provides three-dimensional meridian and acupoint information to develop an artificial intelligence robot for application of acupuncture, acupressure, meridian massage, and so on.

### Technical Solutions

To accomplish the above-mentioned objects, according to a first aspect of the present invention, there is provided a three-dimensional acupoint mapping method including the steps of:

(a) retrieving, by a processor, a standard template having a 3D data of human organ and a standard position of an acupoint;

(b) generating an instance of the standard template in a coordinate system of a 3D image of a patient, wherein the 3D image of the patient includes 3D data of the patient's organ corresponding to the human organ of the standard template;

(c) transforming the coordinate system of the instance of the standard template; and

(d) calculating a patient's acupoint position with the standard position of the acupoint of the instance of the standard template;

According to the present invention, desirably, the standard position of the acupoint includes a relative-coordinates from center point of the 3D data of human organ.

The standard position of the acupoint further includes ratio of 1) distance from the center point of the 3D data of the human organ to a point where surface of the human organ and a straight line connecting the standard position of the acupoint and the center point of the 3D data of the human organ meet, and 2) distance from the point where surface of the human organ and the straight line meet to the point of the standard position of the acupoint is further stored along with the standard position of the acupoint.

According to the present invention, desirably, at the step (c), the processor moves center point of the 3D data of the human organ to center point of the patient's organ in the 3D image of the patient; and scales the coordinate system of coordinate system of the standard template along x-axis, y-axis and z-axis so that the 3D data of the human organ overlapped with the human organs in the 3d image of the patient.

When ratio of 1) distance from the center point of the patient's organ to a point where surface of the patient's organ and a straight line connecting the calculated patient's acupoint's coordinates and the center point of the patient's organ meet, and 2) distance from the point where surface of the patient's organ and the straight line connecting the calculated patient's acupoint's coordinates and the center point of the patient's organ meet to the calculated patient's acupoint's coordinates does not meet the ratio included in the standard position of the acupoint.

The processor compensates the calculated patient's acupoint's coordinates by scaling 2) distance from the point where surface of the patient's organ and the straight line connecting the calculated patient's acupoint's coordinates and the center point of the patient's organ meet to the calculated patient's acupoint's coordinates.

According to a second aspect of the present invention, there is provided a three-dimensional acupoint mapping system including one or more microprocessors; and a non-transitory computer-readable medium or media comprising one or more sequences of instructions which, when executed by the one or more processors, causes steps to be performed comprising:

(a) retrieving, by a processor, a standard template having a 3D data of human organ and a standard position of an acupoint;

(b) generating an instance of the standard template in a coordinate system of a 3D image of a patient, wherein the 3D image of the patient includes 3D data of the patient's organ corresponding to the human organ of the standard template;

(c) transforming the coordinate system of the instance of the standard template; and

(d) calculating a patient's acupoint position with the standard position of the acupoint of the instance of the standard template;

wherein the standard position of the acupoint includes a relative-coordinates from center point of the 3D data of human organ;

and wherein at the step (c), the processor moves center point of the 3D data of the human organ to center point of the patient's organ in the 3D image of the patient; and scales the coordinate system of coordinate system of the standard template along x-axis, y-axis and z-axis so that the 3D data of the human organ overlapped with the human organs in the 3d image of the patient.

To accomplish the above-mentioned objects, according to a third aspect of the present invention, there is provided a computer-readable recording medium for recording a program that executes the three-dimensional acupoint mapping method on a computer.

### Advantageous Effects

As described above, the three-dimensional acupoint mapping device and method according to the present invention maps the standard locations of acupoints with the three-dimensional coordinates onto the three-dimensional standard images, thereby providing three-dimensional acupoint locations and meridian pathways in the deep body as well as on the body surface.

Further, the three-dimensional acupoint mapping device and method according to the present invention determines the standard locations of acupoints in consideration of at least one of anatomical structures such as blood vessels, nerves, muscles, and organs of the human body to provide acupuncture information on safe direction and depth of needle insertion on the basis of the locations of the anatomical structures and the acupuncture skill level of the practitioner.

Furthermore, the three-dimensional acupoint mapping device and method according to the present invention utilizes the three-dimensional acupoint information obtained by mapping the three-dimensional coordinates of the acupoints onto the three-dimensional standard image produced on the basis of the human body image, as materials for education and training.

Also, the three-dimensional acupoint mapping device and method according to the present invention emphasizes at least one of the anatomical structures on the basis of the user's input for the emphasizing option menu, so that the real acupuncture can be more safely performed, without any damage on the important anatomical structures such as the blood vessels, organs and nerves around the acupoint. In addition, the three-dimensional acupoint mapping device and method according to the present invention provides the three-dimensional acupoint locations customized to the patient on the basis of the patient's three-dimensional image, thereby performing the acupuncture according to the patient's characteristics.

Additionally, the three-dimensional acupoint mapping device and method according to the present invention makes use of the three-dimensional acupoint coordinates on the body surface and in the body determined in consideration of the states (locations, sizes, and so on) of the anatomical structure, thereby developing a meridian and acupoint diagnostic device capable of easily analyzing various biometric information (blood flow speed, the number of blood cells, ion concentration, concentration of specific protein, and so on) in the deep body as well as on the body surface around acupoints.

Moreover, the three-dimensional acupoint mapping device and method according to the present invention provides the three-dimensional meridian and/or acupoint information customized to user (patient), thereby allowing an artificial intelligence device (robot) to automatically perform diagnostic analysis on meridian and/or acupoint for the user (patient) and perform safe acupuncture for the user (patient).

Also, the three-dimensional acupoint mapping device and method according to the present invention develops a meridian massage device and an acupressure device based on the three-dimensional information on meridians and acupoints, thereby stimulating more accurately the meridians and acupoints of the user (patient).

### BRIEF DESCRIPTION OF THE DRAWINGS

FIG. 1A shows a schematic diagram of a system for implementing one or more aspects of the present disclosure, and

FIG. 1B shows an overall network configuration for implementing one or more aspects of the present disclosure and a remote server.

FIGS. 2A and 2B show a standard three-dimensional image and a user-customized three-dimensional image, which are displayed by the three-dimensional acupoint mapping device according to the present invention;

FIGS. 3A and 3B show a standard three-dimensional image by acupuncture skill level and a user-customized three-dimensional image by acupuncture skill level, which are displayed by the three-dimensional acupoint mapping device according to the present invention;

FIGS. 4A and 4B show a standard three-dimensional image by disease type and a user-customized three-dimensional image by disease type, which are displayed by the three-dimensional acupoint mapping device according to the present invention;

FIGS. 5A and 5B are views showing an example of a first image displayed by the three-dimensional acupoint mapping device according to the present invention;

FIG. 6 is a view showing an example of a second image displayed by the three-dimensional acupoint mapping device according to the present invention;

FIGS. 7A and 7B are views showing an example of a third image displayed by the three-dimensional acupoint mapping device according to the present invention;

FIGS. 8A-8C are views showing an example of a fourth image displayed by the three-dimensional acupoint mapping device according to the present invention;

FIGS. 9A-9C show an example of determining the location of GB20 by the three-dimensional acupoint mapping device according to the present invention;

FIGS. 10A-10C show an example of determining the location of BL40 by the three-dimensional acupoint mapping device according to the present invention;

FIGS. 11A-11C show an example of determining the location of GB25 by the three-dimensional acupoint mapping device according to the present invention;

FIGS. 12A-12B show an example of determining the location of CV12 by the three-dimensional acupoint mapping device according to the present invention;

FIG. 13 is a conceptual diagram illustrating 3D data of a human organ and a standard position of an acupoint.

FIG. 14 shows a process of overlapping 3D data of an human organ with graphic data of a patient's organ.

FIG. 15 shows an example for explaining the process of transforming the coordinate system of a standard template and calibrating a patient's acupoint coordinates.

FIG. 16 is a flowchart showing a three-dimensional acupoint mapping method according to the present invention.

### DETAILED DESCRIPTION OF THE PREFERRED EMBODIMENTS

Now, an explanation on a three-dimensional acupoint mapping device and method according to the present invention will be given with reference to the attached drawings. Before the present invention is disclosed and described, it is to be understood that the disclosed embodiments are merely exemplary of the invention, which can be embodied in various forms. Therefore, specific structural and functional details disclosed herein are not to be interpreted as limiting.

When real acupuncture is performed on specific acupoints located close to major organs of the human body, much attention is paid to avoid damages of the major organs of the human body, and so as to perform safe acupuncture, accordingly, the direction and depth of needle insertion should be determined in consideration of the tissues (blood vessels, organs, nerves, and muscles as anatomical structures) in the human body and the acupuncture skill level of a practitioner. So as to prevent the principal tissues of the human body from being damaged due to a wrong acupuncture and thus to perform the acupuncture in a more safe manner, the present invention relates to a technology that determines three-dimensional locations of acupoints in consideration of the locations of anatomical structures of the human body to display the location of the determined acupoint in association with a three-dimensional image and that also provides the information for safe acupuncture in consideration of the acupuncture skill level of a practitioner and the precision of an acupuncture robot.

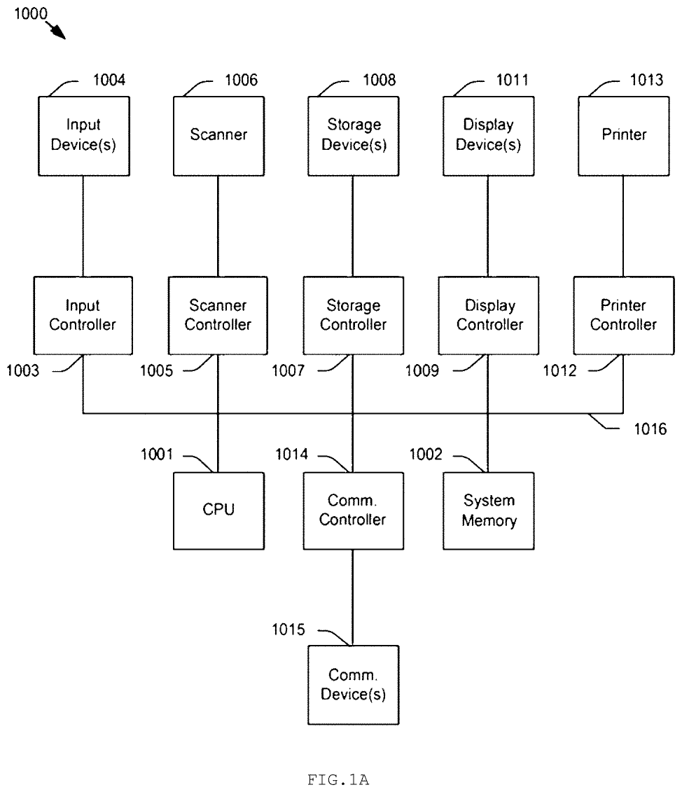

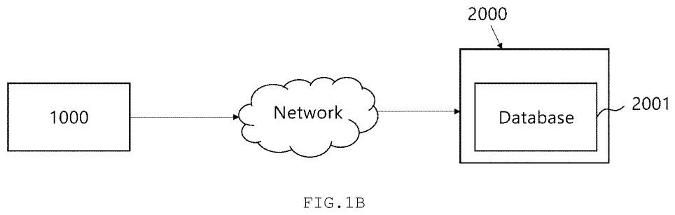

FIG. 1A shows a schematic diagram of a system 1000 for implementing one or more aspects of the present disclosure. It will be understood that the functionalities shown for system 1000 may operate to support various embodiments of the electronic devices (such as mobile devices, servers and satellites) shown in FIG. 1A although it shall be understood that an electronic device may be differently configured and include different components. As illustrated in FIG. 1A, system 1000 includes a central processing unit (CPU) 1001 that provides computing resources and controls the computer. CPU 1001 may be implemented with a microprocessor or the like, and may also include a graphics processor and/or a floating point coprocessor for mathematical computations. System 1000 may also include a system memory 1002, which may be in the form of random-access memory (RAM) and read-only memory (ROM).

A number of controllers and peripheral devices may also be provided, as shown in FIG. 1A. An input controller 1003 represents an interface to various input device(s) 1004, such as a keyboard, mouse, or stylus. There may also be a peripheral controller(s) 1005 represents an interface to various peripheral device(s) 1006, such as a scanner, laser printer or 3D printer. System 1000 may also include a storage controller 1007 for interfacing with one or more storage devices 1008 each of which includes a storage medium such as magnetic tape or disk, or an optical medium that might be used to record programs of instructions for operating systems, utilities and applications which may include embodiments of programs that implement various aspects of the present invention. Storage device(s) 1008 may also be used to store processed data or data to be processed in accordance with the invention. System 1000 may also include a display controller 1009 for providing an interface to a display device 1011, which may be a cathode ray tube (CRT), a thin film transistor (TFT) display, or other type of display. A communications controller 1014 may interface with one or more communication devices 1015, which enables system 1000 to connect to remote devices through any of a variety of networks including the Internet, an Ethernet cloud, a local area network (LAN), a wide area network (WAN), a storage area network (SAN) or through any suitable electromagnetic carrier signals including infrared signals.

In the illustrated system, all major system components may connect to a bus 1016, which may represent more than one physical bus. However, various system components may or may not be in physical proximity to one another. For example, input data and/or output data may be remotely transmitted from one physical location to another. In addition, programs that implement various aspects of this invention may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices.

Embodiments of the present invention may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present invention may further relate to computer products with a non-transitory, tangible computer-readable medium that have computer code thereon for performing various computer-implemented operations. The media and computer code may be those specially designed and constructed for the purposes of the present invention, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present invention may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present invention. One skilled in the art will also recognize that a number of the elements described above may be physically and/or functionally separated into sub-modules or combined together.

If a device fetches stored commands to sequentially perform the commands through a processor, the device is not limited in a specific type of hardware. The device is provided as a type of a smartphone, a tablet PC, a desktop PC, or the like, thereby being performed in a stand-alone manner, and otherwise, the device is provided as a type of a server, thereby transmitting processed results to remote clients connected thereto through network.

Hereinafter the system 1000 will be referred to as a “three-dimensional acupoint mapping system 1000”.

According to FIG. 1B, the three-dimensional acupoint mapping system 1000 is connected to the remote server 2000. The remote server 2000 has a database 2001 that stores standard templates.

In the example of FIG. 1B, the database 2001 is provided in the remote server 2000. However, the database may also be provided in the storage device 1008.

Database 2001 stores standard templates which contain 3D data of human organs and standard positions of acupuncture points.

3D data of human organs are graphic data created by modeling one or more of various organs inside a human body in 3D. One standard template may contain on or more 3D data of human organs. The standard position of acupoint can be expressed as relative positions from 3D data of human organs as explained below.

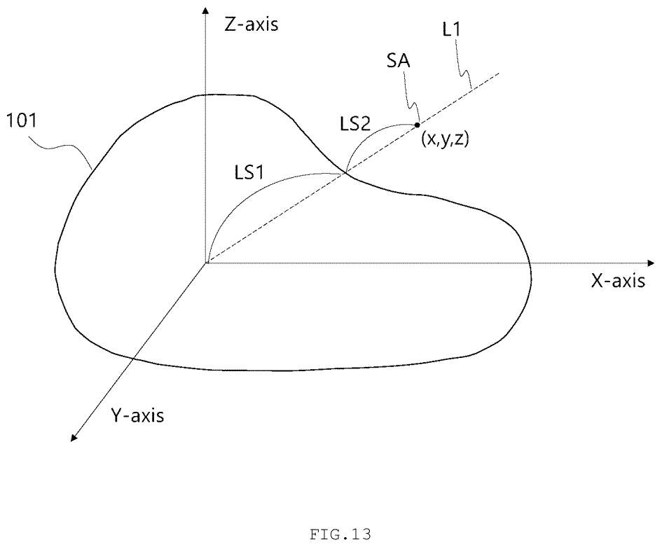

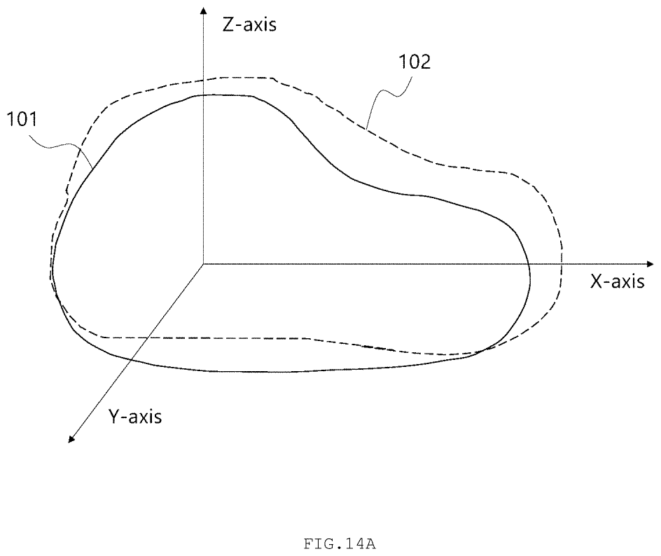

FIG. 13 illustrates 3D data of a human organ and a standard position of acupoint, which can be included in a standard template.

The standard position of the acupoint is a data structure containing relative coordinates from the center point of the 3D data of human organ in a standard template.

The center point of the coordinate system shown in FIG. 13 corresponds to the center point of the 3D data of the human organ as shown. That is, the coordinates of the center point of 3D data of the human organ in the coordinate system of FIG. 13 is (0, 0, 0). Thus, the coordinates the standard acupoint SA may be expressed as (x, y, x).

Since the coordinates of the center point of the 3D data of the human organ is (0, 0, 0), the coordinates of the standard acupoint SA correspond to the relative coordinates from the center point of the 3D data of the human organ.

The standard position of the acupoint further contains the ratio of the two line segments LS1 and LS2 as shown in FIG. 13.

The straight line L1 shown in FIG. 13 connects the standard position of the acupoint and the center point of the 3D data of the human organ.

LS1 represents the distance from the center point of the 3D data of the human organ to the point where the surface of 3D data of the human organ meets the straight line Ll.

Since the human organ is expressed in 3D data, the surface of the human organ means the outer surface on the 3D data.

The line segment LS2 represents the distance from the point where the surface of the human organ and the straight line L1 meet to the standard position of the acupoint.

Therefore, the standard position of the acupoint can be expressed as a data structure captaining both the coordinates of the acupoint and the ratio of the line segments LS1 and LS2.

The 3D data of human organs may be produced on the basis of human body images (or medical images of human body) inclusive of the information on anatomical structures of the human body.

The 3D data of human organs may contain 2D or 3D graphic data regarding at least one of skin, blood vessels, nerves, muscles, and organs as the anatomical structures of the human body therein.

Further, medical human body images obtained by computed tomography (CT), magnetic resonance imaging (MRI), positron emission tomography (PET), and so on, may be used to produce 3D data of human organs.

Accordingly, information on the anatomical structures such as blood vessels, nerves, tendons, ligaments, muscles, and organs are contained in the standard template, but without being limited thereto, information on all anatomical structures to be considered when three-dimensional coordinates of an acupoint are determined or when acupuncture is performed is contained in the standard template.

At this time, the standard template stored within the database 2001 may be used to determine the position of an acupoint of a patient. If a user (patient)'s 3D image is given, further, the standard template is used to identify an anatomical structure in the user's 3D image.

There may be standard templates stored within the database 2001 such as a liver template, a muscle template, a bone template, and so on.

In the meantime, the 3D image of the patient contains 3D coordinate data of the patient's human organs.

By identifying human organs from the patient's human medical image obtained by CT or MRI, and determining a volume and location occupied by each human organ, each organ can be expressed as coordinates in a three-dimensional coordinate system. Each organ of the patient included in the patient's 3D image does not necessarily have to be 3D modeling data in vector format.

The organs of the patient may be expressed as a raster format 3D modeling data or even a set of coordinates in order to identify which organ corresponds to a specific coordinate within the coordinate system of the 3D image.

A process of identifying a human organ from a human medical image may be manually processed by a doctor or identified by artificial intelligence by well-known algorithm or methods.

The processor 1001 calculates and compensates the position of the acupoints of a patient on the 3D image with the standard template.

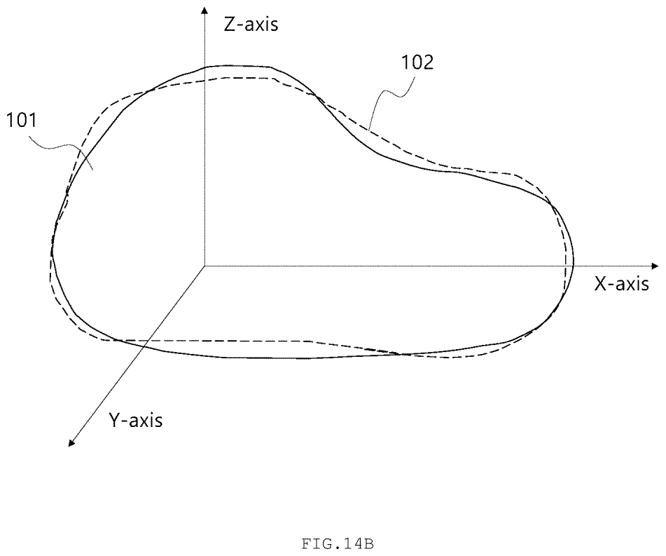

FIG. 14A shows an instance of standard template having 3D data of the human organ 101 corresponding to patient's organ created in the three-dimensional coordinate system of 3D image of the patient.

In FIG. 14A there are two 3D objects 101, 102 visually shown in a three-dimensional coordinate system, but this is a visual representation of the operation process by the processor 1001. This does not mean that these objects have to be displayed on the display device 1011 like FIG. 14.

The processor 1001 creates an instance of the standard template in the 3D coordinate system of the 3D image of the patient. In detail, an instance of the 3D data 101 of the human organ which is included in the standard template is generated, and the standard position of the acupoint included in the standard template is located at a relative position based on the 3D data of the organ 101.

In this case, the processor 1001 moves the center point of the 3D data of the human organ 101 to the center of the coordinate system of the 3D image of the patient.

Meanwhile, since the center point of 3D data of the patient's organ 102 is located at the center of the coordinate system of the patient's 3D image, the center point of both 3D objects 101 and 102 locate at (0, 0, 0).

Therefore, the standard position of acupoint locates at the same coordinates shown in FIG. 13, i.e., (x, y, z).

However, actual shape of the patient's organ will not match the shape of 3D data the human organ 101 of the standard template, which is a virtual 3D image.

In FIG. 14A, the 3D data of the human organ 101 is approximately 10% smaller in the x-axis, 5% smaller in the y-axis direction, and approximately 10% larger in the z-axis direction, compared to the 3D data of the patient's organ 102 which represents the patient's actual shape of the organ.

Accordingly, the processor 1001 adjusts the size of the 3D data of the human organ 101 along the x-axis, along the y-axis, and along the z-axis in order for the volume of the 3D data of the human organ 101 be the same as the volume of the 3D data of the patient's organ 102. More precisely, the processor 1001 scales the coordinate system of the instance of the standard template created in the patient's 3D image along the x-axis, y-axis, and z-axis, respectively.

Desirably, the transformation algorithm may be designed so that the shape of the patient's organ 102 and the 3D data 101 of the human organ overlap each other as much as possible.

To do this, the processor transforms the coordinate system of the instance of the standard template along the x-axis, y-axis, and z-axis gradually, sequentially, repeatedly. When the volume of 3D data of the patient's organ 102 and that of the human organ's 3D data 101 is almost equal within the error range, transformation by the processor is ended.

Typically, in the field of computer software technology related to 3D graphics, transformation of a 3D coordinate system may include one or more of translation, rotation, scaling, and reflection of the 3D coordinate system. In the present invention, moving the center point of the coordinate system by the processor 1001 is referred to as translation, and increasing or decreasing the ratio of the coordinate system is referred to as scaling. Also, the term transformation (or transforming) collectively refers to the procedures like scaling, moving (translation), etc.

FIG. 14B shows that the coordinate system of the instance of the standard template is increased by 10% in the x-axis direction, increased by 5% in the y-axis direction, and reduced by 10% in the z-axis direction.

Thus, the volume of the 3D data of the human organ 101 and the volume of the 3D data of the patient's organ 102 could be the same in the three-dimensional coordinate system.

However, even if the volumes match, the shape of the 3D data of patient's organs 102 will be different form the shape of the 3D data of the human organ 101. In addition, the actual organ of human body does not have a smooth curved surface. Moreover, the shape of the patient's actual organ is simplified in the process of converting the patient's organ into 3D data. Thus, difference between the shape of the patient's actual organ and the 3D data of the human organ 101 will exists.

Calibration should be performed to calculate the patient's acupoints more accurately and to avoid acupuncture on the organs of the human body.

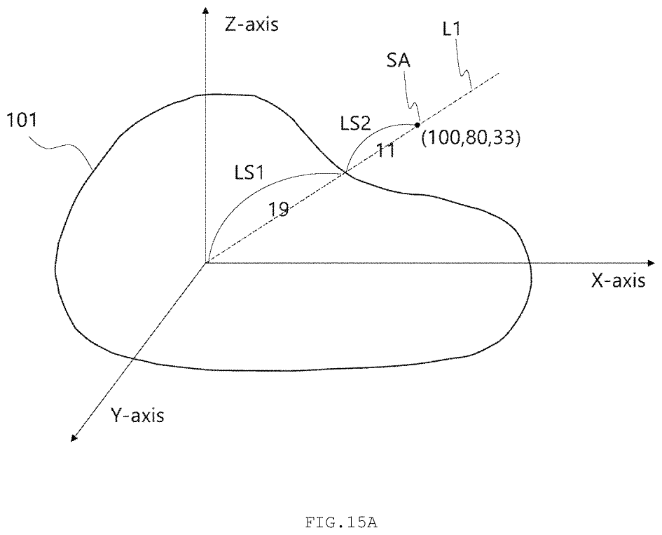

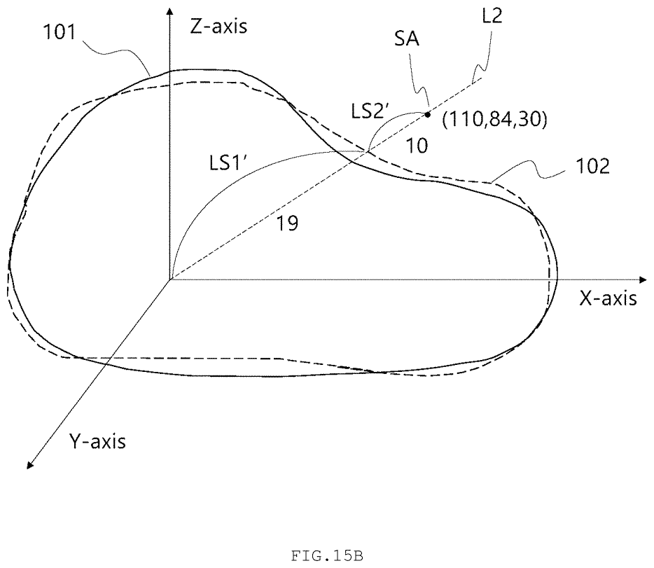

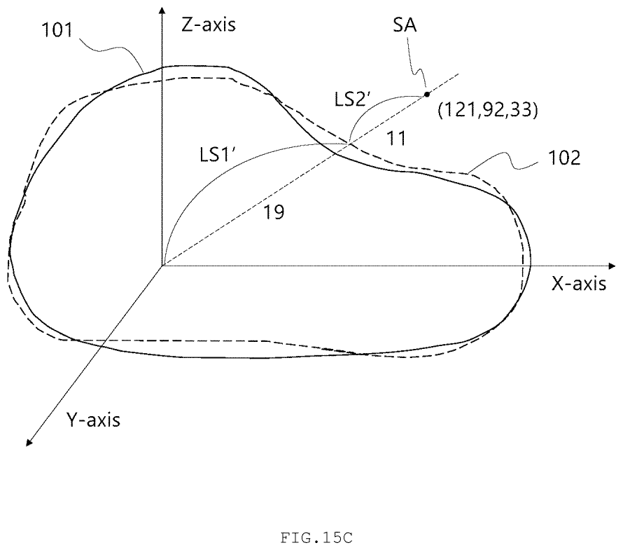

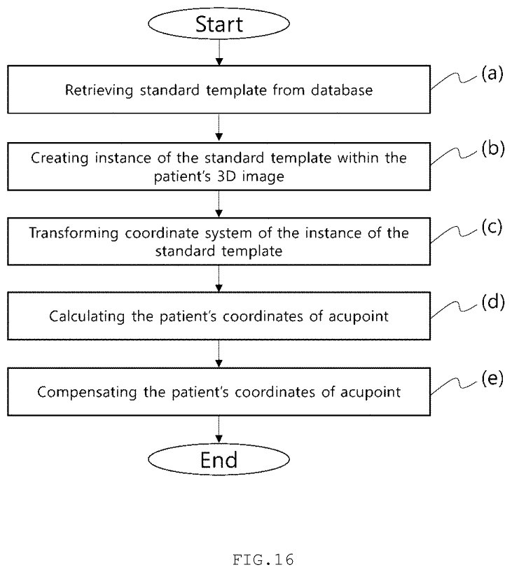

FIG. 15A shows the standard template illustrated in FIG. 13. The coordinates of the standard acupoint SA is (100, 80, 35), and the ratio of LS1 and LS2 is 19:11 according to the example shown in FIG. 15A.

Meanwhile, FIG. 15B shows the result of the transformation process of FIG. 14B. In FIG. 14B, the coordinate system of the instance of the standard template is increased by 10% in the x-axis direction, increased by 5% in the y-axis direction, and shrunk by 10% in the z-axis direction. Thus, the coordinates of standard acupoint SA is transformed to (110, 84, 30).

FIG. 15C shows the result of ratio correction to match the ratio of LS1′ and LS2′ to the ratio of LS1 and LS1.

In FIGS. 15B and 15C, L2 is a straight line connecting the coordinates of the standard acupoint SA of the instance of the transformed standard template and the center of the 3D coordinate system of the 3D image. That is, L2 is a straight line connecting the coordinates of the standard acupoint of the instance of the standard template and the center point of the 3D data of the patient's organ.

That is, in the example of FIG. 15B, L2 is a straight line connecting (0, 0, 0) and (110, 84, 30).

Meanwhile, LS1′ represents the distance from the center of the coordinate system to the point where the straight line L2 and the surface of the 3D data of the patient's organ 102 meet. LS2 represents the distance from the point where the straight line L2 and the surface of 3D data of the patient's organ 102 meet to the standard coordinates of the acupoint of the instance of the transformed standard template.

The ratio of LS1′ and LS2′ is 19:10 as shown in FIG. 15B, which is different from the ratio of LS1 and LS2 of 19:11. Accordingly, while LS1′ left unchanged, the processor 1001 adjusts the length of LS2′ to match the ratio of LS1 and LS2.

Since the size of the patient's organ should be treated as a constant, LS1′ is not subject to calibration.

In this way, the coordinates of the patient's acupoint can be calculated more precisely by reflecting the actual shape of the patient's organs.

The acupoint of the patient calculated with the above-mentioned method can be displayed on a display device 1011.

Furthermore, the processor 1001 displays the three-dimensional pathway of at least one meridian on the body surface and/or in the body on a display device 1011. Accordingly, processor 1001 displays information on the distribution of meridians or locations of acupoints from the body surface to the deep inside the body. Also, the processor 1001 displays the information on the acupoint locations and the meridian pathways on the body surface and/or in the deep body in association with a three-dimensional image of the user (patient) as will be discussed later.

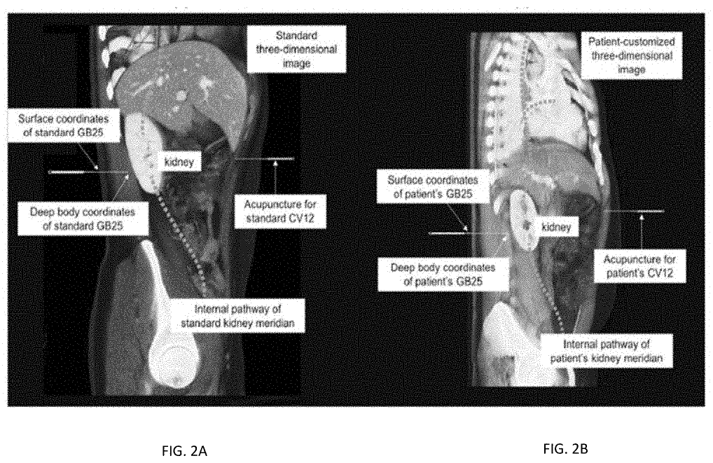

For example, the kidney meridian passes through the kidneys, liver, lungs and heart in the body. As shown in FIGS. 2A-2B, the three-dimensional acupoint mapping system 1000 according to the present invention maps the information on the pathway of the kidney meridian in association with the kidney, liver, lung and heart in the body onto the three-dimensional standard image or the three-dimensional image of the user (patient) and then displays the mapped information. At this time, the three-dimensional acupoint mapping system 1000 according to the present invention displays any one of the three-dimensional standard image or the three-dimensional image of the user (patient), and if necessary, displays both of the three-dimensional standard image and the three-dimensional image of the user (patient).

If it is determined that the input of the user for any one of the displayed three-dimensional acupoint in the body is generated, the processor 1001 displays detailed acupuncture information on the acupoint corresponding to the user's input. In this case, the detailed acupuncture information includes an acupuncture needle icon placed on the acupoint corresponding to the user's input. Further, the detailed acupuncture information includes numerical information on the direction and/or depth of needle insertion into the acupoint corresponding to the user's input. Furthermore, the detailed acupuncture information includes information on the anatomical structure around the acupoint corresponding to the user's input. In relation to the detailed acupuncture information, that is, the processor 1001 displays the acupuncture needle icon placed on the acupoint corresponding to the user's input, displays the information on the direction and/or depth of needle insertion from the surface coordinates of the acupoint to the deep body coordinates of the acupoint corresponding to the user's input, or displays the information on the identification names of the anatomical structures around the acupoint corresponding to the user's input. In this case, the information on the depth of needle insertion indicates depth information from the body surface to a point where the end of a needle reaches, and the information on the direction of needle insertion indicates information on the direction of the needle with respect to the body surface.

Further, the processor 1001 performs at least one of enlargement, reduction and rotation operations for the three-dimensional image in response to an input (for example, drag, separate menu click, or the like) of the user related to image control. At this time, the user's input related to the image control to perform the enlargement, reduction and rotation operations for the three-dimensional image is carried out through previously set drag-and-drop operations in correspondence to respective functions of the image control or through click operations on the icons corresponding to the respective functions of the image control.

Through the operations related to the image control, the processor displays the information on the anatomical structure around the acupoint corresponding to the user's input according to the depths from the body surface to the deep body. That is, the processor displays the three-dimensional image on which the anatomical structure (for example, blood vessel, nerve, muscle, skeleton, and organ) around the acupoint corresponding to the user's input is projected according to the depths from the body surface to the deep body. Particularly, the processor 1001 displays the anatomical structure around the needle inserted into a given acupoint through the three-dimensional image varied according to various depths and angles. Accordingly, the three-dimensional acupoint mapping system 1000 according to the present invention provides accurate information on the three-dimensional coordinates in the body for the acupoint so that safe acupuncture can be performed according to the acupuncture skill level of the practitioner and the precision of the acupuncture robot.

In addition, the processor 1001 displays an emphasizing option menu for emphasizing at least one of the anatomical structures in the three-dimensional image. If the user's input for the emphasizing option menu is generated, at this time, the processor 1001 identifies an element for an emphasizing option corresponding to the user's input and emphasizes and displays the identified element on the three-dimensional image. At this time, examples of the element for the emphasizing option include blood vessels, nerves, muscles, skeleton, and organs, but they are not limited thereto. Through the emphasizing option menu, real acupuncture for the given acupoint can be performed more safely.

For example, the processor 1001 displays ‘blood vessel emphasizing option menu’ for determining whether a blood vessel among the anatomical structures on the three-dimensional image is emphasized or not (YES/NO) on the screen. In this case, if it is determined that the blood vessel is emphasized (YES) by the user's input for the ‘blood vessel emphasizing option menu’, the processor 1001 emphasizes and displays the portion corresponding to the blood vessel on the three-dimensional image displayed on the screen so that the portion is vividly provided for the eyes of the user.

The emphasis on the anatomical structures on the three-dimensional standard image is performed on the basis of the user's input, but it is not limited thereto. On the basis of the information on previously inputted structures with respect to specific acupoints, that is, the previously inputted structures can be automatically emphasized. In this case, the information on the previously inputted structures with respect to the specific acupoints indicates information on the structures to which special attention is paid when the real acupuncture for the specific acupoints is performed.

For example, if a first acupoint is located close to an organ to cause much attention to be paid to the real acupuncture for the first acupoint so as to prevent the organ from being touched by the needle, matching information of the location (three-dimensional coordinates in the body) of the first acupoint with the organ is in advance stored in the three-dimensional acupoint mapping system 1000. If the user's input for the first acupoint is generated, after that, the processor 1001 automatically emphasizes and displays the organ on the three-dimensional standard image on the screen.

Through the emphasis and display of the anatomical structure, the three-dimensional acupoint mapping system 1000 according to the present invention can perform the real acupuncture for the given acupoint more safely. That is, the three-dimensional acupoint mapping system 1000 according to the present invention can emphasize and display at least one (for example, blood vessel, nerve, muscle, skeleton, organ, tendon, ligament, and so on) of the anatomical structures on the three-dimensional image, so that the real acupuncture can be more safely performed, without any damage on the blood vessels, organs and nerves of the user (patient)'s body.

Individual users (patients) have different locations and sizes of their anatomical structure, and so as to provide the locations of the acupoints customized to the individual users, accordingly, there is a need to correct the standard location set with respect to the standard three-dimensional image in consideration of the location and size of the user's anatomical structure. Processes as set-forth below can be done by the processor 1001.

On the three-dimensional standard image, for example, if it is assumed that the surface of the liver is located at a point corresponding to a first distance (for example, 50 mm) from the body surface and the standard location of the first acupoint is placed at a point (corresponding to a distance of 48 mm from the body surface) corresponding to a distance of 2 mm from the surface of the liver within the first distance, it is found that the user's liver is more swollen than the liver on the three-dimensional standard image through the comparison between the user's three-dimensional image and the standard three-dimensional image, and accordingly, it is assumed that the distance from the body surface of the user (patient) to the surface of the liver of the user has a smaller value (for example, 45 mm) than a distance obtained by subtracting the distance of 2 mm from the first distance. At this time, if the real acupuncture for the user is performed on the basis of the standard location of the first acupoint, the needle is inserted into the user's liver because the user (patient)'s liver is swollen, so that the user's liver may be damaged. So as to perform safe acupuncture, accordingly, the three-dimensional acupoint mapping system 1000 according to the present invention can correct the coordinates in the deep body for the first acupoint on the basis of the state of the anatomical structure, that is, the state (location and size) of the liver on the user's three-dimensional. At this time, for example, the coordinates in the deep body for the first acupoint corrected are located at a point corresponding to the distance of 2 mm from the user (patient)'s liver so as to prevent the user (patient)'s liver from being inserted by the needle. The corrected coordinates in the deep body for the first acupoint are used as the user-customized safe acupuncture depth information.

In addition, for example, if it is inappropriate to insert the needle due to obstacles (for example, damages, lesions, foreign bodies, and so on) on the body surface coordinates or between the body surface coordinates and the coordinates in the deep body for the user (patient)'s first acupoint, the body surface coordinates for the first acupoint can be corrected to perform safe acupuncture. The corrected body surface coordinates for the first acupoint are used as the user-customized safe acupuncture direction information.

If the user's input for correction of the acupuncture information of the acupoint is generated, the processor 1001 performs the correction on the safe acupuncture information in consideration of the acupuncture skill level of the practitioner and the precision of the acupuncture robot. At this time, an amount of correction according to the acupuncture skill level of the practitioner and the precision of the acupuncture robot is in advance stored in the three-dimensional acupoint mapping system 1000. For example, if the acupuncture skill level of the practitioner is inputted as a beginning level, the safe acupuncture information is corrected by a first value, and if the acupuncture skill level of the practitioner is inputted as an intermediate level, the safe acupuncture information is corrected by a second value.

As adequate acupuncture methods (directions, depths, speeds, and so on) are different according to diseases, the processor 1001 performs the correction on the acupuncture information on the acupoint in consideration of the kinds of the patient's diseases. At this time, an amount of correction according to the diseases is in advance stored in the three-dimensional acupoint mapping system 1000. For example, if disease A as the patient's disease is inputted, the acupuncture information (depth of needle insertion, direction of needle insertion, speed of needle insertion, and so on) on the first acupoint is corrected by a value of a, and if disease B as the patient's disease is inputted, the acupuncture information on the first acupoint is corrected by a value of b.

At this time, the user-customized three-dimensional acupoint and acupuncture information produced by means of the processor 1001 is displayed on the screen through the processor 1001 in the same manner as the three-dimensional acupoint information in the body based on the three-dimensional standard image.

Further, the three-dimensional acupoint mapping system 1000 according to the present invention performs machine learning through the acupoint location information corrected according to the individual users, and on the basis of the machine learning, the processor 1001 provides the user-customized acupoint location and acupuncture information more accurately.

Under the above-mentioned configuration of the three-dimensional acupoint mapping system 1000, now, an explanation on the examples of the screens on which the three-dimensional acupoint information in the body and/or the user-customized three-dimensional acupoint information in the body produced by the three-dimensional acupoint mapping system 1000 are displayed will be given.

FIGS. 2A and 2B show the examples of the standard three-dimensional image and the user (patient)-customized three-dimensional image displayed by the three-dimensional acupoint mapping system 1000 according to the present invention.

Referring to FIG. 2A, the processor 1001 displays (or provides) the three-dimensional standard image on the screen. At this time, the processor 1001 displays the three-dimensional acupoint information in the body based on the three-dimensional standard image.

For example, the processor 1001 maps and provides the standard location in the deep body (that is, standard coordinates in the deep body) for GB25. Further, the processor 1001 provides the standard location of the body surface (that is, standard coordinates on the body surface) for GB25 in association with the three-dimensional standard image. Furthermore, the processor 1001 displays the acupuncture needle icon located on the corresponding GB25 on the screen on the basis of the standard coordinates on the body surface for GB25 and the standard coordinates in the deep body for GB25. Also, the processor 1001 provides the standard acupuncture information (inclusive of the information on the standard coordinates on the body surface, the standard coordinates in the deep body, standard direction of needle insertion, standard depth of needle insertion, standard speed of needle insertion, and so on) for one acupoint (for example, GB25). In addition, the processor 1001 displays acupuncture information on two or more of acupoints (for example, GB25 and CV12). As mentioned above, further, the processor 1001 displays internal and/or external meridian pathway information inclusive of the three-dimensional coordinate information on the body surface or in the deep body on the three-dimensional standard image. For example, FIG. 2A shows an example where the internal pathway of the kidney meridian flowing in association with the kidney, liver, lung and heart in the body is mapped onto the three-dimensional standard image and displayed.

As shown in FIG. 2B, the processor 1001 displays (or provides) the user (patient)'s three-dimensional image on the screen. At this time, the processor 1001 displays the user-customized three-dimensional acupoint information in the body based on the user's three-dimensional image. For example, the processor 1001 maps the coordinates in the deep body for the user's GB25 (that is, the patient's GB25 coordinates in the deep body) determined on the basis of the state of the anatomical structure of the user's three-dimensional image onto the user's three-dimensional image. In this case, the user's GB25 coordinates in the deep body are determined by correcting the standard location of GB25 in the deep body in consideration of the state of the anatomical structure (for example, the location and size of the kidney) of the user's three-dimensional image through the comparison between the standard three-dimensional image and the user's three-dimensional image.

Further, the processor 1001 provides the user-customized B25 standard coordinates on the body surface (that is, the patient's GB25 coordinates on the body surface) customized to the user in association with the user's three-dimensional image. Further, the processor 1001 displays the acupuncture needle icon located on the corresponding GB25 on the screen on the basis of the user's GB25 coordinates on the body surface and the user's GB25 coordinates in the deep body. Also, the processor 1001 provides the acupuncture information (inclusive of information on the coordinates on the body surface, the coordinates in the deep body, direction of needle insertion, depth of needle insertion, and so on, which are determined in consideration of the state of the anatomical structure in the patient's body) for an acupoint (for example, GB25), as acupuncture information customized to the user. In addition, the processor 1001 displays acupuncture information on two or more of acupoints (for example, GB25 and CV12). For example, the acupuncture needle icon for CV12 displayed on the user's three-dimensional image indicates the acupuncture needle inserted into patient's CV12 determined according to the patient's state. Further, the processor 1001 displays meridian pathway information inclusive of the three-dimensional coordinate information on the body surface and/or in the deep body on the user's three-dimensional image. For example, FIG. 2B shows an example where the internal pathway of the kidney meridian flowing in association with the kidney, liver, lung and heart of the body is mapped onto the user's three-dimensional image and is displayed.

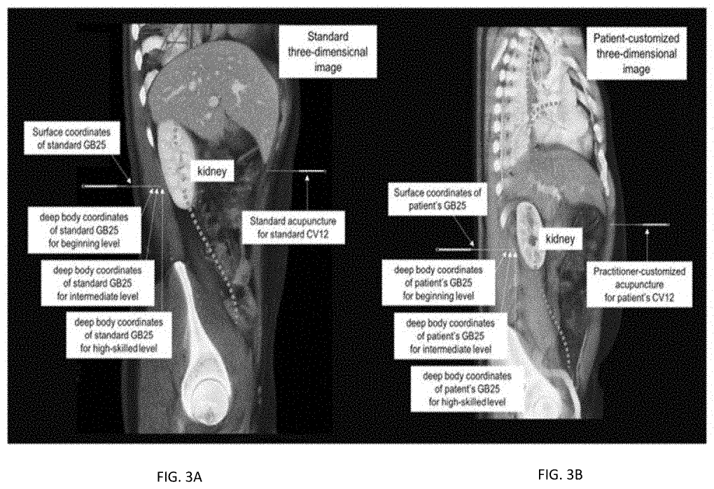

FIGS. 3A and 3B show the examples of safe acupuncture information by acupuncture skill level of practitioner on the standard three-dimensional image and the user-customized three-dimensional image displayed by the three-dimensional acupoint mapping system 1000 according to the present invention. For example, the processor 1001 provides safe acupuncture information for GB25 determined according to the state of the anatomical structure (the location and size of the kidney) and the acupuncture skill level of practitioner (e.g. beginning level, intermediate level, high-skilled level) on the standard three-dimensional image or the user-customized three-dimensional image.

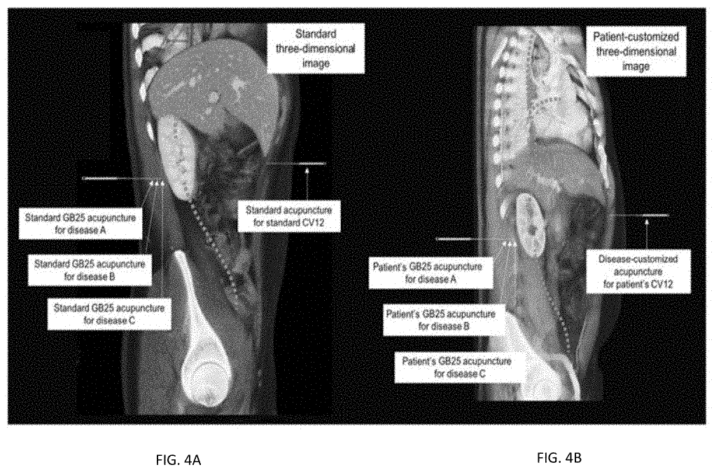

FIGS. 4A and 4B show the examples of acupuncture information by disease type on the standard three-dimensional image and the user-customized three-dimensional image displayed by the three-dimensional acupoint mapping system 1000 according to the present invention. For example, the processor 1001 provides acupuncture information for GB25 determined according to the disease type inputted on the standard three-dimensional image or the user-customized three-dimensional image.

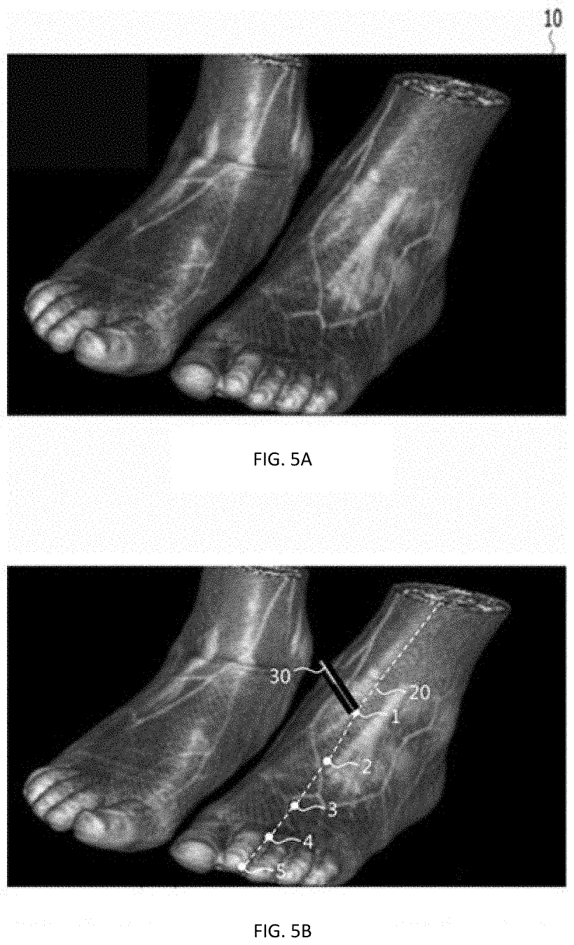

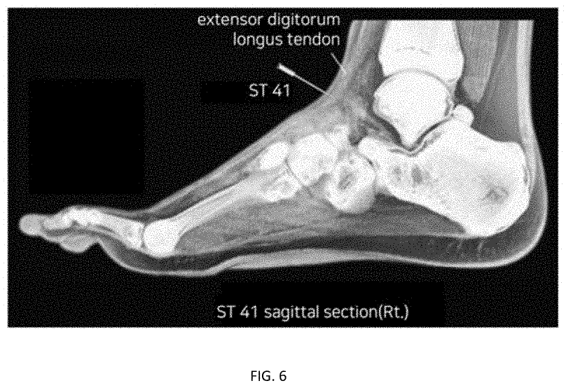

FIGS. 5A and 5B are views showing an example of a first image displayed by the three-dimensional acupoint mapping system 1000 according to the present invention.

As shown in FIGS. 5A and 5B, the processor 1001 displays the three-dimensional image 10 on the screen.

At this time, the three-dimensional image 10 as shown in FIGS. 5A and 5B is the three-dimensional standard image or the user's three-dimensional image. As shown in FIGS. 5A and 5B, the three-dimensional image 10 for feet as a portion of the user's body is displayed.

As shown in FIG. 5B, the processor 1001 maps a meridian pathway 20 including the location information on first to fifth acupoints 1 to 5 onto the three-dimensional image 10 and displays the mapped result. At this time, the acupoint location information and/or the meridian pathway information provided by the three-dimensional acupoint mapping system 1000 according to the present invention are information on the three-dimensional coordinates on the body surface and in the body.

As shown in FIG. 5B, the first acupoint 1 indicates the acupoint corresponding to ST41, the second acupoint 2 the acupoint corresponding to ST42, the third acupoint 3 the acupoint corresponding to ST43, the fourth acupoint 4 the acupoint corresponding to ST44, and the fifth acupoint 5 the acupoint corresponding to ST45. If the user's input for the first acupoint 1 among the first to fifth acupoints is generated, as shown in FIG. 5B, an acupuncture needle icon inserted into the first acupoint 1 is displayed in association with the three-dimensional image 10.

Further, the processor 1001 displays a sectional image of the three-dimensional image for the location of a specific acupoint. In this case, the processor 1001 also displays an acupuncture needle icon inserted into the specific acupoint on the three-dimensional image. Further, the processor 1001 displays images having various sections like sagittal section, transverse section, and so on according to the user's input. An example of the sectional image display is shown in FIG. 6.

FIG. 6 is a view showing an example of a second image (e.g., sectional image) displayed by the three-dimensional acupoint mapping system 1000 according to the present invention.

FIG. 6 shows the sagittal sectional image with the needle inserted into the first acupoint 1 (that is, the needle inserted into ST41) as shown in FIG. 5B. For example, the location of ST41 (inclusive of the location on the body surface and in the body) and acupuncture on ST41 (inclusive of the information on the depth and direction of needle insertion) are determined as the three-dimensional coordinates in the body with respect to the location of an extensor digitorum logus tendon as an anatomical structure.

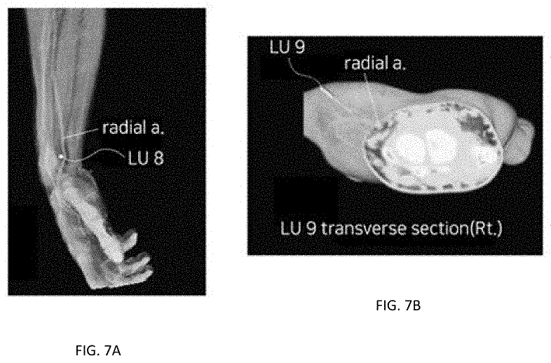

FIGS. 7A and 7B are views showing an example of a third image displayed by the three-dimensional acupoint mapping system 1000 according to the present invention.

As shown in FIGS. 7A and 7B, the three-dimensional acupoint mapping system 1000 according to the present invention determines the standard locations of LU8 and LU9 with respect to the radial artery. Further, as shown in FIG. 7B, the three-dimensional acupoint mapping system 1000 according to the present invention displays the determined standard location of LU9 through a sectional image in consideration of the state of the anatomical structure (e.g., the radial artery) in the body.

As shown in FIGS. 7A and 7B, further, the three-dimensional acupoint mapping system 1000 emphasizes and displays the radial artery on the three-dimensional image so as to prevent the radial artery from being damaged upon the acupuncture. Accordingly, the three-dimensional acupoint mapping system 1000 guides safe acupuncture into the corresponding acupoint on the basis of the three-dimensional image on which the blood vessel is emphasized.

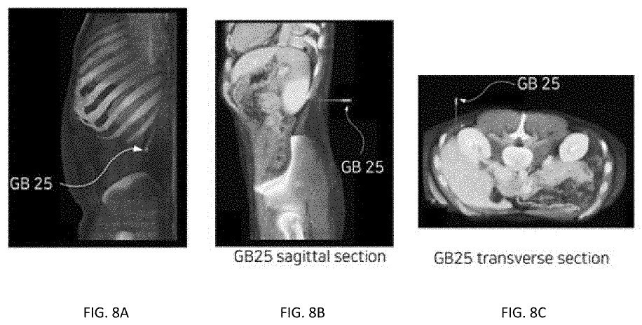

FIGS. 8A-8C are views showing an example of a fourth image displayed by the three-dimensional acupoint mapping system 1000 according to the present invention.

FIG. 8A shows the acupoint GB25 displayed on a CT image. The acupoint GB25 is located over the kidney, and when acupuncture is performed on GB25, attention should be paid to avoid damaging the kidney. Accordingly, the three-dimensional acupoint mapping system 1000 according to the present invention determines the three-dimensional coordinates of GB25 in the deep body in consideration of the location of the kidney.

Referring especially to FIG. 8B showing the transverse sectional image of the acupoint GB25 and to FIG. 8C showing the sagittal sectional image of the acupoint GB25,

the three-dimensional acupoint mapping system 1000 according to the present invention displays the determined acupoint location and the acupuncture needle icon located on the corresponding acupoint in consideration of the location of the anatomical structure in the body on the three-dimensional image, so that the accurate location of the kidney under the acupoint GB25 is provided for the user, and without any damage on the kidney upon the acupuncture, accordingly, the needling depth and direction for safe acupuncture are three-dimensionally provided for the user.

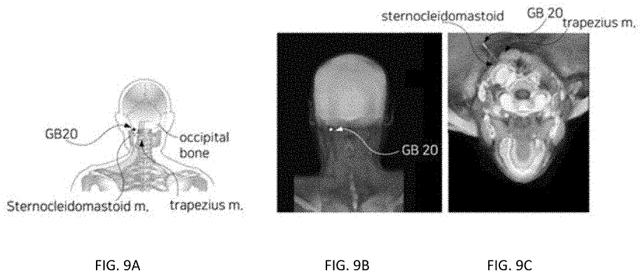

FIGS. 9A-9C show an example of determining the standard location of GB20 by the three-dimensional acupoint mapping system 1000 according to the present invention.

As shown in FIGS. 9A-9C, GB20 is located in the anterior region of the neck, inferior to the occipital bone, in the depression between the origins of sternocleidomastoid muscle and the trapezius muscle.

Also, GB20 is used to treat various diseases such as stroke, eye pain, nosebleed, common cold, insomnia, stiff neck, and so on. The depth and direction of needle insertion on this point depend on the type of disease to be treated.

For example, the direction of needle insertion on GB20 includes a perpendicular insertion, an oblique insertion (needle insertion toward the opposite side eyeball), and a transverse insertion (transverse needle insertion toward right side at left GB20 and toward left side at right GB20), and the depth of needle insertion at GB20 depends on diseases to be treated or the condition of the patient.

So as to determine the standard location (that is, the three-dimensional coordinates) of GB20, the three-dimensional acupoint mapping system 1000 according to the present invention produces the standard templates of the occipital bone, sternocleidomastoid muscle, and trapezius muscle on the three-dimensional standard image by means of the standard template database 110. After that, the mapping unit 120 determines the three-dimensional X, Y and Z coordinates in the body as the standard location of GB20 on the basis of the occipital bone, sternocleidomastoid muscle, and trapezius muscle as the anatomical structures corresponding to the standard templates.

If the three-dimensional image of the patient is inputted, also, the processor 1001 of the three-dimensional acupoint mapping system 1000 according to the present invention applies the standard templates (that is, the standard occipital bone template, the standard sternocleidomastoid muscle template, and the standard trapezius muscle template) produced on the basis of the standard three-dimensional image to the patient's three-dimensional image. Accordingly, the processor 1001 identifies the occipital bone, sternocleidomastoid muscle, and trapezius muscle as the anatomical structures of the patient from the three-dimensional image of the patient.

Next, processor 1001 compares the standard three-dimensional image and the patient's three-dimensional image. Otherwise, processor 1001 compares the standard templates and the anatomical structures identified from the patient's three-dimensional image. For example, the standard occipital bone template is compared with the occipital bone identified from the patient's three-dimensional image.

The individual patients have different locations or sizes of their anatomical structure, and accordingly, processor 1001 calculates a difference between the states of the two anatomical structures through the comparison between the two images or between the two anatomical structures. On the basis of the difference between the states of the two anatomical structures, after that, the processor 1001 corrects the X, Y and Z coordinates as the standard location of GB20 to X′, Y′ and Z′ coordinates according to the states of the anatomical structures of the patient. At this time, the direction and depth of needle insertion depend on diseases, and accordingly, amounts of correction of GB20 are varied according to the diseases of the patient in advance inputted. For example, if a disease A is inputted as the disease information of the patient, the processor 1001 corrects the coordinates of GB20 to X′a, Y′a and Z′a, and if a disease B is inputted as the disease information of the patient, the processor 1001 corrects the coordinates of GB20 to X′b, Y′b and Z′b. Like this, the three-dimensional acupoint mapping system 1000 according to the present invention provides the individualized and customized acupuncture information based on the disease to be treated.

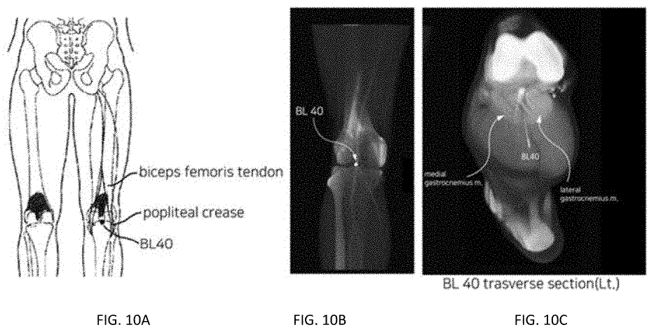

FIGS. 10A-10C show an example of determining the location of BL40 by the three-dimensional acupoint mapping system 1000 according to the present invention.

BL40 is located on the posterior aspect of the knee, at the midpoint of the popliteal crease between the biceps femoris tendon and the semitendinosus tendon.

Also, BL40 is used to treat various diseases such as lumbago, epistaxis, fever, skin ulcer, difficult urination, and so on. The acupuncture technique for this point varies according to the diseases to be treated and the conditions of the patient.

When acupuncture is performed on BL40, attention should be paid to avoid damaging the popliteal artery or tibial nerve. When a pricking bloodletting technique (that is, a therapeutic method to let out a small amount of blood) is applied to this point, the acupuncture needle pricks the popliteal vein. In this case, the needle is inserted and withdrawn at a rapider speed than a general speed.

To perform acupuncture on BL40, the mapping unit 120 of the three-dimensional acupoint mapping system 1000 according to the present invention determines the standard location of BL40 (the standard three-dimensional X, Y and Z coordinates of BL40,) on the basis of the locations of the anatomical structures on the standard three-dimensional image and provides the standard acupuncture information (direction, depth, and speed of needle insertion) so that upon the acupuncture, the needle does not come into contact with the popliteal artery and the tibial nerve.

After that, the processor 1001 determines the location of patient's BL40 (the patient-customized three-dimensional X′, Y′ and Z′ coordinates) on the basis of the anatomical structures such as biceps femoris tendon and semitendinosus tendon on the patient's three-dimensional image.

Further, the processor 1001 identifies the popliteal artery from the patient's three-dimensional image with the popliteal artery template produced on the basis of the standard three-dimensional image, and with the identified result, the acupuncture information for patient's BL40 is corrected through the processor 1001. If the patient's BL40 coordinates X′, Y′ and Z′ come into contact with the popliteal artery identified from the patient's three-dimensional image or pass therethrough, the processor 1001 corrects the coordinates of patient's BL40 to X′a, Y′a and Z′a on the basis of the location of the popliteal artery as the anatomical structure of the patient, so that upon the acupuncture of BL40, the needle does not prick the popliteal artery.

When a pricking bloodletting technique is applied to BL40, the processor 1001 corrects the coordinates of patient's BL40 to X′b, Y′b and Z′b so as to allow the needle to come into contact with the popliteal vein, and then corrects the speeds of needle insertion and withdrawal, thereby providing the adequate acupuncture information with which the pricking bloodletting is achieved on BL40. At this time, the processor 1001 emphasizes and displays the popliteal vein as one of the anatomical structures contained in the patient's three-dimensional image. Like this, the three-dimensional acupoint mapping system 1000 according to the present invention provides the user-customized acupuncture information based on the treatment purpose and the acupuncture technique.

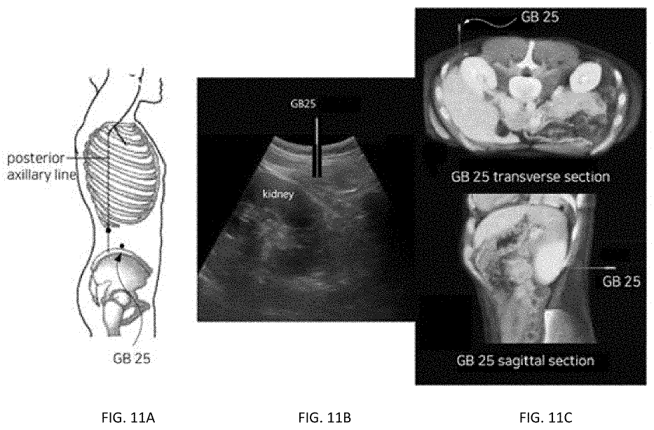

FIGS. 11A-11C is a view showing an example of determining the location of GB25 by the three-dimensional acupoint mapping system 1000 according to the present invention.

As shown in FIGS. 11A-11C, GB25 is located on the lateral abdomen, inferior to the free extremity of the 12th rib. The free extremity of the 12th rib can be palpated below the inferior border of the costal arch posterior to the posterior axillary line.

The direction and depth of needle insertion into GB25 depends on the disease to be treated or the condition of the patient. Further, there is the kidney in the deep portion under GB25, and when acupuncture is performed on GB25, accordingly, attention should be paid to avoid damaging the kidney.

The mapping unit 120 determines the standard X, Y and Z coordinates of GB25, on the standard three-dimensional image on the basis of the standard templates for the anatomical structures such as the free extremity of the 12th rib, the kidney, and so on.

If the patient's three-dimensional image is inputted, after that, processor 1001 compares the patient's three-dimensional image with the standard three-dimensional image, and the processor 1001 corrects the coordinates of GB25 to patient-customized X′, Y′ and Z′ coordinates on the basis of the correction condition in advance set to prevent the kidney from being pricked by the needle in consideration of the anatomical structures of the patient. For example, the processor 1001 corrects the coordinates of GB25 to the coordinates corresponding to a location distant by a given distance from the surface of the kidney of the patient or a location corresponding to a given percentage of a distance from the surface of the body of the patient to the surface of the kidney of the patient.

Further, the processor 1001 corrects the safe acupuncture information in consideration of the acupuncture skill level of the practitioner. If the acupuncture practitioner is at a beginning level, for example, the processor 1001 corrects the coordinates to X′a, Y′a and Z′a corresponding to a location distant by a distance ‘a’ from the surface of the kidney of the patient or a location corresponding to 70% of a distance from the surface of the body of the patient to the surface of the kidney of the patient, if he or she is at an intermediate level, the processor 1001 corrects the coordinates to X′b, Y′b and Z′b corresponding to a location distant by a distance ‘b’ from the surface of the kidney of the patient or a location corresponding to 80% of a distance from the surface of the body of the patient to the surface of the kidney of the patient, and if he or she is at a high skilled level, the processor 1001 corrects the coordinates to X′c, Y′c and Z′c corresponding to a location distant by a distance ‘c’ from the surface of the kidney of the patient or a location corresponding to 90% of a distance from the surface of the body of the patient to the surface of the kidney of the patient. Like this, the three-dimensional acupoint mapping system 1000 according to the present invention provides the practitioner-customized safe acupuncture information based on the acupuncture skill level of the acupuncture practitioner.

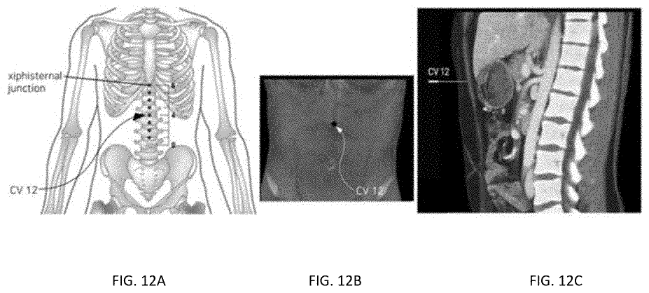

FIGS. 12A-12C show an example of determining the location of CV12 by the three-dimensional acupoint mapping system 1000 according to the present invention.

As shown in FIGS. 12A-12C, CV12 is located on the upper abdomen, on the anterior median line, at the midpoint of the line connecting the xiphisternal junction and the center of umbilicus.

Some of patients have the swollen liver or the liver located at a lower place than a general place, and accordingly, the acupuncture on CV12 is performed with carefulness.

The mapping unit 120 determines the standard three-dimensional location of CV12 (standard X, Y and Z coordinates of CV12,) on the standard three-dimensional image on the basis of the xiphisternal junction and the umbilicus.

If the patient's three-dimensional image is inputted, after that, processor 1001 compares the patient's three-dimensional image with the standard three-dimensional image, and the processor 1001 performs the correction on the location of CV12 according to the disease and the condition of the patient in advance inputted.

The individual patients have different locations of the liver, and the three-dimensional acupoint mapping system 1000 according to the present invention identifies the location of the liver from the patient's three-dimensional image on the basis of the standard liver template. After that, if the patient's liver is more swollen or located at a lower place than the standard liver template, the processor 1001 corrects the three-dimensional location of CV12 on the basis of the correction condition in advance set. For example, if the patient's CV12 coordinates X′, Y′ and Z′ are within an area corresponding to the liver of the patient's three-dimensional image, the processor 1001 previously sets conditions such as a location distant by distance ‘a’ from the surface of the liver of the patient or a location corresponding to percentage ‘a’ of a distance from the surface of the body of the patient to the surface of the liver of the patient and then corrects the coordinates to X′a, Y′a and Z′a, so that upon real acupuncture for CV12, the needle insertion into the patient's liver can be prevented. Like this, the three-dimensional acupoint mapping system 1000 according to the present invention provides the individualized and customized acupuncture information based on the state and constitution of the patient.

The three-dimensional acupoint mapping system 1000 according to the present invention utilizes the information on the three-dimensional acupoints and meridian pathways in the body as materials for education, and also provides really measured human body materials for the three-dimensional acupoints and meridian pathways in the body. Further, the three-dimensional acupoint mapping system 1000 according to the present invention provides the information on the three-dimensional acupoint locations (the three-dimensional coordinates) on the body surface and in the deep body. Accordingly, the three-dimensional acupoint mapping system 1000 provides the acupuncture information inclusive of the direction and depth of needle insertion.

Also, the three-dimensional acupoint mapping system 1000 according to the present invention inputs the three-dimensional medical image information of the user (patient) to the standard templates to provide the user-customized acupoint location information. Accordingly, the present invention utilizes the three-dimensional coordinates of acupoints in the body as clinical assistant, thereby improving the accuracy and safety of the acupuncture.

Further, the three-dimensional coordinates of acupoints in the body determined in consideration of the states of the anatomical structures in the body, which are provided by the three-dimensional acupoint mapping system 1000 according to the present invention, are used to analyze physiological and pathological states (that is, various biometric information inclusive of blood flow speed, the number of blood cells, ion concentration, and concentration of specific protein) in the deep portions of the acupoints as well as on the body surface of the acupoints, and are also used as traditional Korean medicine diagnostic information.

Also, the three-dimensional coordinates in the body for the acupoints provided by the three-dimensional acupoint mapping system 1000 according to the present invention allow an artificial intelligence acupuncture device (or robot) to perform the acupuncture on the given acupoint more accurately and safely. Furthermore, a massage or acupressure device (or robot) for domestic use or commercial use can be developed with the information on the three-dimensional coordinates of acupoints and meridians in the body provided by the three-dimensional acupoint mapping system 1000 according to the present invention

Further, the three-dimensional mapping system 1000 according to the present invention is applicable to animals as well as human bodies.

Mode for the Invention

On the basis of the above-mentioned three-dimensional mapping system 1000, hereinafter, an explanation on a three-dimensional mapping method according to the present invention will be briefly given.

FIG. 16 is a flowchart showing a three-dimensional acupoint mapping method according to the present invention.

As shown in FIG. 16, the three-dimensional acupoint mapping method according to the present invention is carried out by means of the three-dimensional acupoint mapping system 1000. Even if not explained, the contents explained on the three-dimensional acupoint mapping system 1000 will be applied to the three-dimensional acupoint mapping method in the same manner.

Referring to FIG. 16, at step (a), the processor 1001 retrieves a standard template that have a 3D data of human organ 101 and a standard position of an acupoint SA from database 2001.

As discussed above, the standard position of the acupoint SA is a data-structure containing a relative-coordinates from center point of the 3D data of the human organ in the coordinate system of the standard template. The standard position of the acupoint may also contain ratio of L1 and L2 as shown in FIG. 13.

At step (b), the processor 1001 generates an instance of the standard template in a coordinate system of a 3D image of a patient when a 3D image of the patient including 3D data of the patient's organ 102 corresponding to the human organ of the standard template is input.

The processor 1001 moves center point of the 3D data of the human organ 101 to center point of the 3D data of the patient's organ 102; and scales the coordinate system of the instance of the standard template along x-axis, y-axis and z-axis so that volume of the 3D data of the human organ 101 and the 3D data of the patient's organ 102 to be the same.

Then, at step (c), the processor 1001 transforms the coordinate system of the instance of the standard template. At step (d), the processor 1001 calculates a patient's acupoint's coordinates (or, equivalently patient's acupoint position) with the standard position of the acupoint of the instance of the standard template.

If ratio of LS1′ and LS2′ does not equal to the ratio of LS1 and LS2, the processor 1001 performs the step (e).

At the step (e), the processor 1001 compensates the calculated patient's acupoint's coordinates by scaling LS2′ to make ratio of LS1′ and LS2′ to be equal to the ratio of LS1 and LS2.

The three-dimensional acupoint mapping method according to the present invention is carried out in a form of program commands executed by various computer means and is recorded in computer readable media. The computer readable media include program commands, data files, and data structures solely or combinedly.

Further, the three-dimensional acupoint mapping method according to the present invention is carried out to the form of a computer program or application stored in the recording media and executed by the computer.

What is claimed is:

1. A computer-implemented method for mapping an acupoint in a three-dimensional (3D) coordinate system, comprising:
(a) retrieving, by a processor, a standard template having a 3D data of human organ and a standard position of an acupoint;
(b) generating an instance of the standard template in a coordinate system of a 3D image of a patient, wherein the 3D image of the patient includes 3D data of the patient's organ corresponding to the human organ of the standard template;
(c) transforming the coordinate system of the instance of the standard template; and
(d) calculating a patient's acupoint position with the standard position of the acupoint of the instance of the standard template;
wherein the standard position of the acupoint represents a location relative to a center point of the 3D data of the human organ in the transformed coordinate system of the instance of the standard template;
and wherein the step (c) includes moving the center point of the 3D data of the human organ to a center point of the 3D data of the patient's organ and scaling the coordinate system of the instance of the standard template along x-axis, y-axis and z-axis so that a volume of the 3D data of the human organ is same as a volume of the 3D data of the patient's organ.

2. The computer-implemented method of claim 1, wherein the standard position of the acupoint further contains a first ratio between 1) a distance from the center point of the 3D data of the human organ to a point where a surface of the human organ in the 3D data of the human organ meets a straight line that connects the standard position of the acupoint to the center point of the 3D data of the human organ, and 2) a distance from the point where the surface of the human organ in the 3D data of the human organ meets the straight line to the point of the standard position of the acupoint.

3. The computer-implemented method of claim 2, further comprising:
(e) calculating a second ratio between 1) a distance from the center point of the 3D data of the patient's organ to a point where a surface of the human organ in the 3D data of the patient's organ meets a straight line that connects the patient's acupoint position to the center point of the 3D data of the patient's organ, and 2) a distance from the point where the surface of the human organ in the 3D data of the patient's organ meets the straight line connecting the patient's acupoint position to the center point of the 3D data of the patient's organ; and
f) responsive to a difference between the first and second ratios, compensating the patient's acupoint position by scaling the distance from the point where the surface of the human organ in the 3D data of the patient's organ meets the straight line connecting the patient's acupoint position to the center point of the 3D data of the patient's organ.

4. A system for mapping an acupoint in a three-dimensional (3D) coordinate system, comprising:
one or more microprocessors; and
a non-transitory computer-readable medium or media comprising one or more sequences of instructions which, when executed by the one or more processors, causes steps to be performed comprising:
(a) retrieving a standard template having a 3D data of human organ and a standard position of an acupoint;
(b) generating an instance of the standard template in a coordinate system of a 3D image of a patient, wherein the 3D image of the patient includes 3D data of the patient's organ corresponding to the human organ of the standard template;
(c) transforming the coordinate system of the instance of the standard template; and
(d) calculating a patient's acupoint position with the standard position of the acupoint of the instance of the standard template;
wherein the standard position of the acupoint represents a location relative to a center point of the 3D data of the human organ in the transformed coordinate system of the instance of the standard template;
and wherein the step (c) includes moving the center point of the 3D data of the human organ to a center point of the 3D data of the patient's organ and scaling the coordinate system of the instance of the standard template along x-axis, y-axis and z-axis so that a volume of the 3D data of the human organ is same as a volume of the 3D data of the patient's organ.

5. A non-transitory computer-readable medium or media comprising one or more sequences of instructions which, when executed by one or more processors, causes steps for mapping an acupoint in a three-dimensional (3D) coordinate system comprising:
(a) retrieving a standard template having a 3D data of human organ and a standard position of an acupoint;
(b) generating an instance of the standard template in a coordinate system of a 3D image of a patient, wherein the 3D image of the patient includes 3D data of the patient's organ corresponding to the human organ of the standard template;
(c) transforming the coordinate system of the instance of the standard template; and
(d) calculating a patient's acupoint position with the standard position of the acupoint of the instance of the standard template;
wherein the standard position of the acupoint represents a location relative to a center point of the 3D data of the human organ in the transformed coordinate system of the instance of the standard template;
and wherein the step (c) includes moving the center point of the 3D data of the human organ to a center point of the 3D data of the patient's organ and scaling the coordinate system of the instance of the standard template along x-axis, y-axis and z-axis so that a volume of the 3D data of the human organ is same as a volume of the 3D data of the patient's organ.
